(19) United States
(12) Patent Application Publication
(10) Pub. No.: US 2022/0170003 A1
(43) Pub. Date: Jun. 2, 2022

(54) Means And Methods For Improving Protease Expression

(71) Applicant: Novozymes A/S, Bagsvaerd (DK)

(72) Inventors: Ane Quesada Ganuza, Vitoria (SP); Michael Dolberg Rasmussen, Vallensbaek (DK); Allan Kent Nielsen, Copenhagen (DK)

(73) Assignee: Novozymes A/S, Bagsvaerd (DK)

(21) Appl. No.: 17/437,201

(22) PCT Filed: Mar. 4, 2020
(86) PCT No.: PCT/EP2020/055609

§ 371 Date: Sep. 8, 2021

(30) Foreign Application Priority Data
Mar. 8, 2019 (EP) 19161661.4

(51) Int. Cl. C12N 9/54; C12N 1/20; C12N 9/90

(52) U.S. Cl. CPC C12N 9/54; C12N 1/20; C12Y 502/01008; C12Y 304/21; C12N 9/90; C12Y 304/21062

(57) ABSTRACT

The present invention relates to means and methods for improving protease expression by co-expression with a foldase.

### REFERENCE TO A SEQUENCE LISTING

This application contains a Sequence Listing in computer-readable form, which is incorporated herein by reference.

### FIELD OF THE INVENTION

The present invention relates to means and methods for improving protease expression by co-expression with a foldase.

### BACKGROUND OF THE INVENTION

Within industrial biotechnology, there is a continuous need for improving production yield and thereby process profitability in the production of enzymes and other industrially relevant proteins. A successful strategy has been to employ production host cells that over-express the gene encoding the target protein, e.g., by using multicopy strains containing several gene copies or enhancing the activity of the gene by modifying its control sequences. To fully leverage the beneficial effects of gene over-expression, it would be desirable to increase the secretory capacity of the production host cell in order to overcome any bottlenecks in the secretory machinery.

Foldases are proteins that assist in folding of other proteins. Over-expression of one or more foldases in a production host cell may provide an enhanced folding of a given target protein, which in turn is likely to result in enhanced secretion of correctly folded protein and thereby an improved production yield.

PrsA is an extracytoplasmic foldase identified in various Gram-positive bacteria, including the industrially relevant Bacillus licheniformis. PrsA is a dimeric lipoprotein anchored in the outer leaflet of the cell membrane, where it aids folding of proteins secreted via the conserved SecA-YEG pathway.

Over-expression of native PrsA was shown to improve expression of polypeptides in Gram-positive bacteria (WO 1994/019471).

We have observed that co-expression of the Bacillus clausii alkaline protease (AprH) with certain bacterial foldases results in markedly improved expression of AprH. Based on this finding, we propose that these specific foldases may be useful for improving expression of proteases in general.

### SUMMARY OF THE INVENTION

The present invention relates to the surprising and inventive finding that co-expression of Bacillus clausii alkaline protease (AprH) and certain bacterial foldases provides an improved expression yield of AprH.

In a first aspect, the present invention relates to a nucleic acid constructs comprising:

a) a first heterologous promoter operably linked to at least one polynucleotide encoding a protease; and

b) a second heterologous promoter operably linked to at least one polynucleotide encoding a foldase;

wherein the foldase has a sequence identity of at least 80% to SEQ ID NO: 6 or SEQ ID NO: 9.

In a second aspect, the present invention relates to expression vectors comprising a nucleic acid construct according to the first aspect.

In a third aspect, the present invention relates to Gram-positive host cells comprising in the genome a nucleic acid construct according to the first aspect and/or an expression vector according to the second aspect.

In a fourth aspect, the present invention relates to methods for producing a protease, the methods comprising:

a) providing a Gram-positive host cell according to the third aspect;

b) cultivating said host cell under conditions conducive for expression of the protease; and, optionally,

c) recovering the protease.

### BRIEF DESCRIPTION OF THE FIGURES

FIG. 1 shows a phylogenetic tree depicting the interrelationship between different PrsA homologs obtained from various Gram-positive species. Branch lengths are proportional to the divergence of the amino acid sequence. PrsA from Bacillus sp. is number 25 and has 47.7% sequence identity to PrsA from B. subtilis. PrsA from Geobacillus caldoxylosilyticus is number 26 and has 53.3% sequence identity to PrsA from B. subtilis.

FIG. 2 shows a schematic view of the linear DNA product used for integration of a prsA gene in B. subtilis strain AN2.

FIG. 3 shows a schematic view of the linear DNA product used for integration of the aprH gene in B. subtilis strains AN2, AN2406, and AN2407.

### DEFINITIONS

Foldase: The term “foldase” means an enzyme having foldase activity. Foldase are proteins that facilitate folding of polypeptides into a functional three-dimensional structure, and/or prevent aggregation of unfolded polypeptides into non-functional structures and any subsequent proteolytic degradation. PrsA is an example of a foldase in Gram-positive bacteria. PrsA is a dimer consisting of two monomers that forms two domains; a peptidylprolyl isomerase (PPIase, E.C. 5.2.1.8) domain that interconverts the cis and trans isomers of peptidyl-prolyl bonds, and a foldase domain that assists polypeptide folding (Jakob et al., 2015, J. Biol. Chem. 290(6): 3278-3292). A crystal structure of PrsA from B. subtilis is provided in Jakob et al., supra.

Foldase activity: The term “foldase activity” means PPIase activity and/or foldase activity and is determined as the expression yield or the activity yield of a polypeptide, e.g., a protease, of interest upon co-expression of this polypeptide with a foldase in a suitable host cell. Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Clade: The term “Glade” means a group of polypeptides clustered together on the basis of homologous features traced to a common ancestor. Polypeptide clades can be visualized as phylogenetic trees and a Glade is a group of polypeptides that consists of a common ancestor and all its lineal descendants. Polypeptides forming a group within the Glade (a subclade) of the phylogenetic tree can also share common properties and are more closely related than other polypeptides in the Glade.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a protease and a polynucleotide encoding a foldase of the present invention. Each control sequence may be native (i.e., from the same gene) or foreign (i.e., from a different gene) to the polynucleotide encoding the protease and/or the polynucleotide encoding the foldase or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide of the invention. The term “heterologous promoter” means a promoter that is foreign (i.e., from a different gene) to the polynucleotide to which it is operably linked.

Expression: The term “expression” includes any step involved in the production of a protease including, but not limited to, transcription, post-transcriptional modification, translation, post-translational modification, and secretion.

Expression vector: The term “expression vector” means a linear or circular DNA molecule that comprises polynucleotide of the invention and is operably linked to control sequences that provide for their expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature protease or a polynucleotide that encodes a mature foldase, depending on the context.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Variant: The term “variant” means a polypeptide having protease activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more (e.g., several) amino acids, e.g., 1-5 amino ac-ids, adjacent to and immediately following the amino acid occupying a position.

### SEQUENCE LISTING

SEQ ID NO: 1: Polynucleotide sequence of B. clausii alkaline protease (AprH).

SEQ ID NO: 2: Polypeptide sequence of B. clausii alkaline protease (AprH) including signal peptide.

SEQ ID NO: 3: Mature polypeptide sequence of B. clausii alkaline protease (AprH).

SEQ ID NO: 4: Polynucleotide sequence encoding PrsA consisting of the signal peptide of B. subtilis PrsA and the mature polypeptide of Bacillus sp. PrsA.

SEQ ID NO: 5: Polypeptide sequence of PrsA consisting of the signal peptide of B. subtilis PrsA and the mature polypeptide of Bacillus sp. PrsA.

SEQ ID NO: 6: Mature polypeptide sequence of Bacillus sp. PrsA.

SEQ ID NO: 7: Polynucleotide sequence encoding PrsA consisting of the signal peptide of B. subtilis PrsA and the mature polypeptide of Geobacillus caldoxylosilyticus PrsA.

SEQ ID NO: 8: Polypeptide sequence of PrsA consisting of the signal peptide of B. subtilis PrsA and the mature polypeptide of Geobacillus caldoxylosilyticus PrsA.

SEQ ID NO: 9: Mature polypeptide sequence of Geobacillus caldoxylosilyticus PrsA.

SEQ ID NO: 10: Polynucleotide sequence of sigF gene.

SEQ ID NO: 11: Polynucleotide sequence of sigF Δ297 bp.

SEQ ID NO: 12: SOE PCR product for integration of the prsA gene from Bacillus sp. in AN2.

SEQ ID NO: 13: SOE PCR product for integration of the prsA gene from Geobacillus caldoxylosilyticus in AN2.

SEQ ID NO: 14: SOE PCR product for integration of the aprH gene from Bacillus clausii in AN2, AN2406, and AN2407.

### DETAILED DESCRIPTION OF THE INVENTION

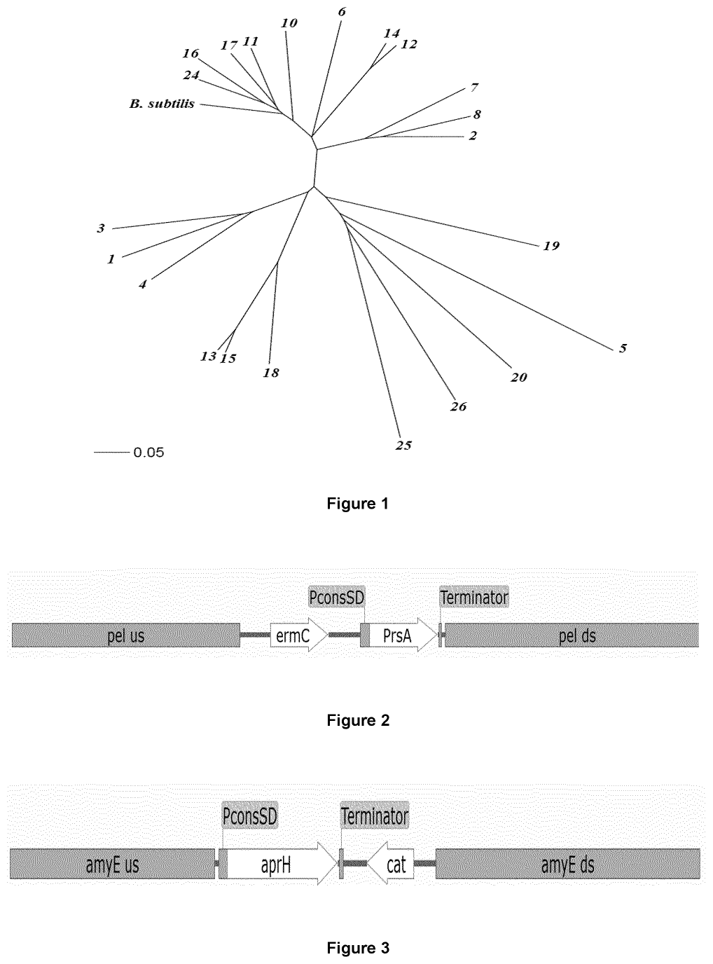

The present invention relates to the surprising and inventive finding that co-expression of the Bacillus clausii alkaline protease (AprH) and certain bacterial foldases provides an improved expression yield of AprH. Upon cultivation of Bacillus subtilis strains co-expressing AprH (SEQ ID NO: 3) together with either Bacillus sp. PrsA (SEQ ID NO: 6) or Geobacillus caldoxylosilyticus PrsA (SEQ ID NO: 9), the expression yield of AprH was increased 14% and 23%, respectively. Moreover, a phylogenetic analysis revealed that these particular foldases are closely related (FIG. 1), suggesting that other structurally related foldases, e.g., foldases belonging to the same phylogenetic Glade, will have similar beneficial effects on AprH expression.

Based on this finding, we propose that Bacillus sp. PrsA and Geobacillus caldoxylosilyticus PrsA as well as closely related foldases are useful for improving expression of proteases in general, in particular serine proteases such as subtilisins.

Nucleic Acid Constructs

In a first aspect, the present invention relates to a nucleic acid construct comprising:

a) a first heterologous promoter operably linked to at least one polynucleotide encoding a protease; and

b) a second heterologous promoter operably linked to at least one polynucleotide encoding a foldase;

wherein the foldase has a sequence identity of at least 80% to SEQ ID NO: 6 or SEQ ID NO: 9.

The first and second heterologous promoter may be any heterologous promoter suitable for expression of the protease and foldase, respectively. In an embodiment, the first heterologous promoter and the second heterologous promoter are same or different promoter; preferably the first heterologous promoter and the second heterologous promoter are identical copies of the same promoter.

The nucleic acid constructs of the invention comprise at least one (i.e., one or more, e.g., 1, 2, 3, 4, 5, 6, 7, 8, 9, 10, or more) polynucleotide encoding a protease. In some embodiments, the nucleic acid constructs of the invention comprise two or more polynucleotides encoding two or more proteases, wherein the two or more protease are the same or different protease.

The protease may be any protease, e.g., a microbial, plant, animal, or human protease. Preferably, the protease is secreted. Preferably, the protease is a serine protease, cysteine protease, threonine protease, aspartic protease, glutamic protease, metalloprotease, or asparagine peptide lyase. More preferably, the protease is a serine protease; even more preferably a subtilase; most preferably a subtilisin.

In some embodiments, the protease comprises a C- or N-terminal propeptide and/or an N-terminal signal peptide. In some embodiments, the protease is a mature protease.

In a preferred embodiment, the protease has a sequence identity of at least 80%, e.g., at least 85%, at least 90%, at least 91%, at least 92%, at least 93%, at least 94%, at least 95%, at least 96%, at least 96%, at least 97%, at least 98%, at least 99%, or 100%, to SEQ ID NO: 3. More preferably, the protease comprises or consists of SEQ ID NO: 3. Most preferably, the protease is Bacillus clausii alkaline protease (AprH) or a variant thereof.

Preferably, the at least one polynucleotide encoding a protease has a sequence identity of at least 80%, e.g., at least 85%, at least 90%, at least 91%, at least 92%, at least 93%, at least 94%, at least 95%, at least 96%, at least 96%, at least 97%, at least 98%, at least 99%, or 100%, to the mature polypeptide coding sequence of SEQ ID NO: 1. More preferably, the at least one polynucleotide encoding a protease comprises or consists of SEQ ID NO: 1.

The nucleic acid constructs of the invention further comprise at least one (i.e., one or more, e.g., 1, 2, 3, 4, 5, 6, 7, 8, 9, 10, or more) polynucleotide encoding a foldase. The foldase has a sequence identity of at least 80%, e.g., at least 85%, at least 90%, at least 91%, at least 92%, at least 93%, at least 94%, at least 95%, at least 96%, at least 96%, at least 97%, at least 98%, at least 99%, or 100%, to SEQ ID NO: 6 or SEQ ID NO: 9. Preferably, the foldase comprises or consists of SEQ ID NO: 6 or SEQ ID NO: 9. Most preferably, the foldase is Bacillus sp. PrsA or a variant thereof or Geobacillus caldoxylosilyticus PrsA or a variant thereof.

Preferably, the at least one polynucleotide encoding a foldase has a sequence identity of at least 80%, e.g., at least 85%, at least 90%, at least 91%, at least 92%, at least 93%, at least 94%, at least 95%, at least 96%, at least 96%, at least 97%, at least 98%, at least 99%, or 100%, to the mature polypeptide coding sequence of SEQ ID NO: 4 or SEQ ID NO: 7. Preferably, at least one polynucleotide encoding a foldase comprises or consists of SEQ ID NO: 4 or SEQ ID NO: 7.

In some embodiments, the nucleic acid constructs of the invention comprise two or more polynucleotides encoding two or more foldases, wherein the two or more foldases are the same or different foldase, i.e., Bacillus sp. PrsA (SEQ ID NO: 6) and/or Geobacillus caldoxylosilyticus PrsA (SEQ ID NO: 9). Thus, in some embodiments, the two or more polynucleotides encoding two or more foldases has a sequence identity of at least 80%, e.g., at least 85%, at least 90%, at least 91%, at least 92%, at least 93%, at least 94%, at least 95%, at least 96%, at least 96%, at least 97%, at least 98%, at least 99%, or 100%, to the mature polypeptide coding sequence of SEQ ID NO: 4 and/or SEQ ID NO: 7.

The at least one polynucleotide encoding a protease and the at least one polynucleotide encoding a foldase (i.e., the polynucleotides of the invention) are operably linked to one or more control sequences that direct the expression of the coding sequence in a suitable host cell under conditions compatible with the control sequences. The polynucleotides may be manipulated in a variety of ways to provide for expression of the protease and/or foldase. Manipulation of a polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. Preferably, the promoter is a heterologous promoter. The promoter contains transcriptional control sequences that mediate the expression of the protease and/or foldase. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis crylllA gene (Agaisse and Lereclus, 1994, Molecular Microbiology 13: 97-107), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator sequence is operably linked to the 3′-terminus of the polynucleotides of the invention. Any terminator that is functional in the host cell may be used.

Preferred terminators for bacterial host cells are obtained from the genes for Bacillus clausii alkaline protease (aprH) and Bacillus licheniformis alpha-amylase (amyL).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis ctyIIIA gene (WO 94/25612) and a Bacillus subtilis SP82 gene (Hue et al., 1995, Journal of Bacteriology 177: 3465-3471).

The control sequence may also be a signal peptide coding region that encodes a signal peptide linked to the N-terminus of the protease and/or foldase and directs the protease and/or foldase into the cell's secretory pathway. The 5′-end of the coding sequence of the polynucleotides of the invention may inherently contain a signal peptide coding sequence naturally linked in translation reading frame with the segment of the coding sequence that encodes the protease and/or foldase. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the protease and/or foldase. However, any signal peptide coding sequence that directs the expressed protease and/or foldase into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a protease and/or foldase. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE) or Bacillus subtilis neutral protease (nprT).

Where both signal peptide and propeptide sequences are present, the propeptide sequence is positioned next to the N-terminus of the protease and/or foldase and the signal peptide sequence is positioned next to the N-terminus of the propeptide sequence.

It may also be desirable to add regulatory sequences that regulate expression of the protease and/or foldase relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems.

Polynucleotides

The present invention also relates to a polynucleotide encoding a protease and a polynucleotide encoding a foldase of the invention. In an embodiment, the polynucleotides have been isolated.

The techniques used to isolate or clone a polynucleotide are known in the art and include isolation from genomic DNA or cDNA, or a combination thereof. The cloning of the polynucleotides from genomic DNA can be performed, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Bacillus, or a related organism and thus, for example, may be an allelic or species variant of the polypeptide encoding region of the polynucleotides of the invention.

Expression Vectors

In a second aspect, the present invention also relates to recombinant expression vectors comprising a nucleic acid construct comprising:

(a) a first heterologous promoter operably linked to at least one polynucleotide encoding a protease;

(b) a second heterologous promoter operably linked to at least one polynucleotide encoding a foldase;

wherein the foldase has a sequence identity of at least 80% to SEQ ID NO: 6 or SEQ ID NO: 9.

The expression vectors of the invention also comprise additional control sequences such as transcriptional and translational stop signals.

The various polynucleotides and control sequences may be joined together to produce a recombinant expression vector that may include one or more convenient restriction sites to allow for insertion or substitution of the polynucleotides of the invention at such sites. Alternatively, the polynucleotides of the invention may be expressed by inserting the polynucleotides or a nucleic acid construct comprising the polynucleotides into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance.

The vector preferably contains an element(s) that permits integration of the vector into the host cell's genome or autonomous replication of the vector in the cell independent of the genome.

For integration into the host cell genome, the vector may rely on the coding sequence of the polynucleotides of the invention or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pUB110, pE194, pTA1060, and pAMß1 permitting replication in Bacillus.

More than one copy of a polynucleotides of the invention may be inserted into a host cell to increase production of a protease and/or foldase. An increase in the copy number of a polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

In a third aspect, the present invention also relates to Gram-positive host cells comprising in the genome:

(i) a nucleic acid construct comprising:
a) a first heterologous promoter operably linked to at least one polynucleotide encoding a protease; and
b) a second heterologous promoter operably linked to at least one polynucleotide encoding a foldase;
wherein the foldase has a sequence identity of at least 80% to SEQ ID NO: 6 or SEQ ID NO: 9; and/or

(ii) an expression vector comprising said nucleic acid construct.

A nucleic acid construct and/or expression vector comprising the polynucleotides of the invention is introduced into a host cell so that the construct or vector is maintained as a chromosomal integrant or as a self-replicating extra-chromosomal vector as described earlier.

The term “host cell” encompasses any progeny of a parent cell that is not identical to the parent cell due to mutations that occur during replication. The choice of a host cell will to a large extent depend upon the gene encoding the protease and its source.

The prokaryotic host cell may be any Gram-positive cell useful in the recombinant production of a protease. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces.

The Gram-positive host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278).

The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294).

The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436).

However, any method known in the art for introducing DNA into a host cell can be used.

Methods of Production

In a fourth aspect, the present invention also relates to methods of producing a protease, the method comprising:

I) providing a Gram-positive host cell comprising:

i) a nucleic acid construct comprising:

a) a first heterologous promoter operably linked to at least one polynucleotide encoding a protease; and

b) a second heterologous promoter operably linked to at least one polynucleotide encoding a foldase;

wherein the foldase has a sequence identity of at least 80% to SEQ ID NO: 6 or SEQ ID NO: 9; and/or

ii) an expression vector comprising said nucleic acid contruct;

II) cultivating said Gram-positive host cell under conditions conducive for expression of the protease and the foldase; and, optionally

III) recovering the protease.

Preferably, the Gram-positive host cell is a Bacillus host cell; preferably the Bacillus host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cell.

The Gram-positive host cells are cultivated in a nutrient medium suitable for production of the protease using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the protease to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the protease is secreted into the nutrient medium, the protease can be recovered directly from the medium. If the protease is not secreted, it can be recovered from cell lysates.

The protease may be detected using methods known in the art that are specific for the protease. These detection methods include, but are not limited to, use of specific antibodies, formation of an enzyme product, or disappearance of an enzyme substrate. For example, an enzyme assay may be used to determine the activity of the protease.

The protease may be recovered using methods known in the art. For example, the protease may be recovered from the nutrient medium by conventional procedures including, but not limited to, collection, centrifugation, filtration, extraction, spray-drying, evaporation, or precipitation.

The protease may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure protease.

In an alternative aspect, the protease is not recovered, but rather a Gram-positive host cell of the present invention expressing the protease is used as a source of the variant.

Fermentation Broth Formulations or Cell Compositions

The present invention also relates to a fermentation broth formulation or a cell composition comprising a protease, and optionally a foldase, of the present invention. The fermentation broth product further comprises additional ingredients used in the fermentation process, such as, for example, cells (including the Gram-positive host cells containing the polynucleotides encoding the protease and the foldase of the present invention which are used to produce the protease), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the Gram-positive host cells are grown to saturation, incubated under carbon-limiting conditions to allow protein synthesis. In some embodiments, the cell-killed whole broth or composition contains the spent cell culture medium, extracellular enzymes, and killed Gram-positive cells. In some embodiments, the Gram-positive host cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 1990/15861 or WO 2010/096673.

Enzyme Compositions

The present invention also relates to compositions comprising a protease, and optionally a foldase, of the present invention.

The compositions may comprise a protease of the present invention as the major enzymatic component, e.g., a mono-component composition. Alternatively, the compositions may comprise multiple enzymatic activities, such as one or more (e.g., several) enzymes selected from the group consisting of hydrolase, isomerase, ligase, lyase, oxidoreductase, or transferase; more preferably the enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

### PREFERRED EMBODIMENTS

1) A nucleic acid construct comprising:
a) a first heterologous promoter operably linked to at least one polynucleotide encoding a protease; and
b) a second heterologous promoter operably linked to at least one polynucleotide encoding a foldase;
wherein the foldase has a sequence identity of at least 80% to SEQ ID NO: 6 or SEQ ID NO: 9.

2) The nucleic acid construct according to embodiment 1, wherein the first heterologous promoter and the second heterologous promoter are same or different promoter; preferably the first heterologous promoter and the second heterologous promoter are identical copies of the same promoter.

3) The nucleic acid construct according to any of the preceding embodiments, wherein the protease is a serine protease, cysteine protease, threonine protease, aspartic protease, glutamic protease, metalloprotease, or asparagine peptide lyase.

4) The nucleic acid construct according to embodiment 3, wherein the protease is a serine protease; preferably a subtilase, most preferably a subtilisin.

5) The nucleic acid construct according to any of the preceding embodiments, wherein the protease comprises a C- or N-terminal propeptide and/or an N-terminal signal peptide; or wherein the protease is a mature protease.

6) The nucleic acid construct according to any of the preceding embodiments, wherein the protease has a sequence identity of at least 80% to SEQ ID NO: 3.

7) The nucleic acid construct according to any of the preceding embodiments, wherein the protease comprises or consists of SEQ ID NO: 3.

8) The nucleic acid construct according to any of the preceding embodiments, wherein the protease is Bacillus clausii alkaline protease (AprH) or a variant thereof.

9) The nucleic acid construct according to any of the preceding embodiments, wherein the at least one polynucleotide encoding a protease has a sequence identity of at least 80% to the mature polypeptide coding sequence of SEQ ID NO: 1.

10) The nucleic acid construct according to any of the preceding embodiments, wherein the at least one polynucleotide encoding a protease comprises or consists of SEQ ID NO: 1.

11) The nucleic acid construct according to any of the preceding embodiments, wherein the at least one polynucleotide encoding a foldase has a sequence identity of at least 80% to the mature polypeptide coding sequence of SEQ ID NO: 4 or SEQ ID NO: 7.

12) The nucleic acid construct according to any of the preceding embodiments, wherein the at least one polynucleotide encoding a foldase comprises or consists of SEQ ID NO: 4 or SEQ ID NO: 7.

13) An expression vector comprising a nucleic acid construct comprising:
a) a first heterologous promoter operably linked to at least one polynucleotide encoding a protease; and
b) a second heterologous promoter operably linked to at least one polynucleotide encoding a foldase;
wherein the foldase has a sequence identity of at least 80% to SEQ ID NO: 6 or SEQ ID NO: 9.

14) The expression vector according to embodiment 13, wherein the first heterologous promoter and the second heterologous promoter are same or different promoter; preferably the first heterologous promoter and the second heterologous promoter are identical copies of the same promoter.

15) The expression vector according to any of embodiments 13-14, wherein the protease is a serine protease, cysteine protease, threonine protease, aspartic protease, glutamic protease, metalloprotease, or asparagine peptide lyase.

16) The expression vector according to embodiment 15, wherein the protease is a serine protease; preferably a subtilase, most preferably a subtilisin.

17) The expression vector according to any of embodiments 13-16, wherein the protease comprises a C- or N-terminal propeptide and/or an N-terminal signal peptide; or wherein the protease is a mature protease.

18) The expression vector according to any of any of embodiments 13-17, wherein the protease has a sequence identity of at least 80% to SEQ ID NO: 3.

19) The expression vector according to any of any of embodiments 13-18, wherein the protease comprises or consists of SEQ ID NO: 3.

20) The expression vector according to any of embodiments 13-19, wherein the protease is Bacillus clausii alkaline protease (AprH) or a variant thereof.

21) The expression vector according to any of embodiments 13-20, wherein the at least one polynucleotide encoding a protease has a sequence identity of at least 80% to the mature polypeptide coding sequence of SEQ ID NO: 1.

22) The expression vector according to any of embodiments 13-21, wherein the at least one polynucleotide encoding a protease comprises or consists of SEQ ID NO: 1.

23) The expression vector according to any of embodiments 13-22, wherein the at least one polynucleotide encoding a foldase has a sequence identity of at least 80% to the mature polypeptide coding sequence of SEQ ID NO: 4 or SEQ ID NO: 7.

24) The expression vector according to any of the preceding embodiments, wherein the at least one polynucleotide encoding a foldase comprises or consists of SEQ ID NO: 4 or SEQ ID NO: 7.

25) A Gram-positive host cell comprising in its genome:
(i) a nucleic acid construct comprising:
   a) a first heterologous promoter operably linked to at least one polynucleotide encoding a protease; and
   b) a second heterologous promoter operably linked to at least one polynucleotide encoding a foldase;
   wherein the foldase has a sequence identity of at least 80% to SEQ ID NO: 6 or SEQ ID NO: 9; and/or
(ii) an expression vector comprising said nucleic acid construct.

26) The Gram-positive host cell according to embodiment 25, wherein the Gram-positive host cell is a Bacillus host cell; preferably the Bacillus host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cell.

27) The Gram-positive host cell according to any of embodiments 25-26, wherein the first heterologous promoter and the second heterologous promoter are same or different promoter; preferably the first heterologous promoter and the second heterologous promoter are identical copies of the same promoter.

28) The Gram-positive host cell according to any of embodiments 25-27, wherein the protease is a serine protease, cysteine protease, threonine protease, aspartic protease, glutamic protease, metalloprotease, or asparagine peptide lyase.

29) The Gram-positive host cell according to embodiment 28, wherein the protease is a serine protease; preferably a subtilase, most preferably a subtilisin.

30) The Gram-positive host cell according to any of embodiments 25-29, wherein the protease comprises a C- or N-terminal propeptide and/or an N-terminal signal peptide; or wherein the protease is a mature protease.

31) The Gram-positive host cell according to any of embodiments 25-30, wherein the protease has a sequence identity of at least 80% to SEQ ID NO: 3.

32) The Gram-positive host cell according to any of embodiments 25-31, wherein the protease comprises or consists of SEQ ID NO: 3.

33) The Gram-positive host cell according to any of embodiments 25-32, wherein the protease is Bacillus clausii alkaline protease (AprH) or a variant thereof.

34) The Gram-positive host cell according to any of embodiments 25-33, wherein the at least one polynucleotide encoding a protease has a sequence identity of at least 80% to the mature polypeptide coding sequence of SEQ ID NO: 1.

35) The Gram-positive host cell according to any of embodiments 25-34, wherein the at least one polynucleotide encoding a protease comprises or consists of SEQ ID NO: 1.

36) The Gram-positive host cell according to any of embodiments 25-35, wherein the at least one polynucleotide encoding a foldase has a sequence identity of at least 80% to the mature polypeptide coding sequence of SEQ ID NO: 4 or SEQ ID NO: 7.

37) The Gram-positive host cell according to any of embodiments 25-36, wherein the at least one polynucleotide encoding a foldase comprises or consists of SEQ ID NO: 4 or SEQ ID NO: 7.

38) A method for producing a protease, the method comprising:
I) providing a Gram-positive host cell comprising in its genome:
   i) a nucleic acid construct comprising:
      a) a first heterologous promoter operably linked to at least one polynucleotide encoding the protease; and
      b) a second heterologous promoter operably linked to at least one polynucleotide encoding a foldase;
      wherein the foldase has a sequence identity of at least 80% to SEQ ID NO: 6 or SEQ ID NO: 9; and/or
   ii) an expression vector comprising said nucleic acid construct;
II) cultivating said Gram-positive host cell under conditions conducive for expression of the protease and the foldase; and, optionally
III) recovering the protease.

39) The method according to embodiment 38, wherein the Gram-positive host cell is a Bacillus host cell; preferably the Bacillus host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cell.

40) The method according to any of embodiments 38-39, wherein the first heterologous promoter and the second heterologous promoter are same or different promoter; preferably the first heterologous promoter and the second heterologous promoter are identical copies of the same promoter.

41) The method according to any of embodiments 38-40, wherein the protease is a serine protease, cysteine protease, threonine protease, aspartic protease, glutamic protease, metalloprotease, or asparagine peptide lyase.

42) The method according to embodiment 41, wherein the protease is a serine protease; preferably a subtilase, most preferably a subtilisin.

43) The method according to any of embodiments 38-42, wherein the protease comprises a C- or N-terminal propeptide and/or an N-terminal signal peptide; or wherein the protease is a mature protease.

44) The method according to any of embodiments 38-43, wherein the protease has a sequence identity of at least 80% to SEQ ID NO: 3.

45) The method according to any of embodiments 38-44, wherein the protease comprises or consists of SEQ ID NO: 3.

46) The method according to any of embodiments 38-45, wherein the protease is Bacillus clausii alkaline protease (AprH) or a variant thereof.

47) The method according to any of embodiments 38-46, wherein the at least one polynucleotide encoding a protease has a sequence identity of at least 80% to the mature polypeptide coding sequence of SEQ ID NO: 1.

48) The method according to any of embodiments 38-47, wherein the at least one polynucleotide encoding a protease comprises or consists of SEQ ID NO: 1.

49) The method according to any of embodiments 38-48, wherein the at least one polynucleotide encoding a foldase has a sequence identity of at least 80% to the mature polypeptide coding sequence of SEQ ID NO: 4 or SEQ ID NO: 7.

50) The method according to any of embodiments 38-49, wherein the at least one polynucleotide encoding a foldase comprises or consists of SEQ ID NO: 4 or SEQ ID NO: 7.

The present invention is further described by the following examples that should not be construed as limiting the scope of the invention.

### EXAMPLES

Materials and Methods

Chemicals used as buffers and substrates were commercial products of at least reagent grade.

PCR amplifications were performed using standard textbook procedures, employing a commercial thermocycler and either Ready-To-Go PCR beads, Phusion polymerase, or RED-TAQ polymerase from commercial suppliers.

LB agar: See EP 0 506 780.

LBPSG agar plates contains LB agar supplemented with phosphate (0.01 M K₃PO₄), glucose (0.4%), and starch (0.5%); See EP 0 805 867 B1.

TY (liquid broth medium): See WO 94/14968, p. 16.

Oligonucleotide primers were obtained from Eurofins, Aarhus, Denmark. DNA manipulations (plasmid and genomic DNA preparation, restriction digestion, purification, ligation, DNA sequencing) was performed using standard textbook procedures with commercially available kits and reagents.

DNA was introduced into B. subtilis rendered naturally competent, either using a two-step procedure (Yasbin et al., 1975, J. Bacteriol. 121: 296-304), or a one-step procedure in which cell material from an agar plate was resuspended in Spizisen 1 medium (WO 2014/052630), 12 ml shaken at 200 rpm for approx. 4 hours at 37° C., DNA added to 400 microliter aliquots, and these further shaken at 150 rpm for 1 hour at the desired temperature before plating on selective agar plates.

All of the constructions described in the examples were assembled from synthetic DNA fragments ordered from GeneArt—ThermoFisher Scientific. The fragments were assembled by sequence overlap extension (SOE) as described in the Examples below.

Genomic DNA was prepared from all relevant isolates using the commercially available QIAamp DNA Blood Kit from Qiagen.

Standard Microplate Batch-Fermentation

PrsA library strains were grown in biological triplicates in 500 mL LB media in 96 deep well plates (CR1496b, EnzyScreen), covered with Sandwich Covers (CR1296, EnzyScreen). Cultures were grown for 24 hours at 37° C. and 300 rpm in Clamp Systems (CR1700, EnzyScreen) (1). After 24 hours, samples were taken for enzymatic activity assays.

All assays were performed in 96 microtiter plates and samples were each measured at 2 different dilutions simultaneously against B. clausii alkaline protease (AprH) as standard. Assays were performed on a Biomek Fx liquid handler and absorbance readings were measured on a Spectramax plate reader (Molecular Devices).

Samples were diluted in Dilution buffer (Tris pH 9.0+0.01% Triton X). 20 μl of diluted sample was mixed with substrate solution (0.6 mg/ml Suc-ala-ala-pro-phe-pNA, Bachem) in dilution buffer. Kinetic absorbance at 405 nM was measured immediately for 5 min and results were extrapolated from the corresponding standard curve.

Strains

| Strain | pel locus | amyE locus |
|---|---|---|
| Bacillus subtilis 168 | — | — |
| (Kunst et al. 1997) | | |
| AN2 (B. subtilis | — | — |
| 168; ΔsigF) | | |
| AN2406 | Bacillus sp. PrsA | — |
| AN2407 | Geobacillus | — |
| | caldoxylosilyticus | |
| | PrsA | |
| AQG88 | — | AprH |
| AQG812 | Geobacillus | AprH |
| | caldoxylosilyticus | |
| | PrsA | |
| AQG825 | Bacillus sp. PrsA | AprH |

Construction of Phylogenetic Tree

The pylogenetic tree depicted as FIG. 1 was constructed by performing a ClustalW alignment (Bioconductor 3.8, “msa” package, R), followed by the constructed of an identity matrix (dist.alignment function in R, W. M. Fitch, s.I.: J. Mol. Biol., vol. 16, pp. 9-16) and a neighbor-joining tree estimation (nj function in R, N. Saitou and M. Nei, s.I.: Molecular Biology and Evolution, vol. 4, pp. 406-425). The tree was plotted as an unrooted tree.

### Example 1. Construction of the B. subtilis Host AN2

B. subtilis AN2 was used as a host strain for expression of prsA and protease genes as described in the following examples. AN2 is a sporulation deficient derivative of B. subtilis 168 due to deletion of 297 bp in the sigF gene (the full sigF gene sequence is provided as SEQ ID NO: 10 and the inactive version containing the deletion is provided as SEQ ID NO: 11).

### Example 2. Construction of Expression Cassettes for prsA Genes and Chromosomal Integration of these in B. subtilis AN2

The B. subtilis strain AN2 was used as a host strain for insertion of expression cassettes for copies of the prsA gene. PrsA expression cassettes were integrated into the pel locus and consisted of the synthetic promoter P_{consSD} followed by a prsA gene and the B. subtilis prsA native terminator. DNA for integrations can be assembled by PCR amplifications of synthetic DNA consisting of the following DNA components: pel 5′ region+ermC (conferring resistance to erythromycin)+synthetic consensus promoter with SD sequence (PconsSD)+prsA open reading frame with terminator+pel 3′ region. The purified PCR products were used in a subsequent PCR reaction to create a single linear DNA by the Gene Splicing by Overlapping Extension (SOE) method (Horton RM 1989) and the Phusion Hot Start DNA Polymerase system (Thermo Scientific) as follows: The PCR amplification reaction mixture contained 50 ng of each of the gel purified PCR products and a thermocycler was used to assemble and amplify the DNA. The resulting SOE product was used directly for transformation of the B. subtilis host AN2. Chromosomal integration was facilitated by homologous recombination and cells wherein double cross-over events occurred were selected for on LB agar plates containing 1 μg/ml erythromycin. A schematic view of a linear DNA product used for integration of a prsA gene in AN2 is shown in FIG. 2 and the sequence of the DNA used for integration of the prsA gene from Bacillus sp. in AN2 resulting in strain AN2406 is provied as SEQ ID NO: 12. A strain expressing the prsA gene from Geobacillus caldoxylosilyticus (DNA sequence provided as SEQ ID NO: 13) was constructed by a similar process and named AN2407.

### Example 3. Construction of an Expression Cassette for the B. clausii Alkaline Protease Gene (aprH) and Chromosomal Integration of this in B. subtilis Strains AN2, AN2406 and AN2407

An aprH expression cassette was integrated into the amyE loci of B. subtilis AN2, AN2406, and AN2407 and consisted of the synthetic promoter P_{conSD} followed by the aprH gene and the B. amyloliquefaciens amyQ terminator. The amyE gene becomes inactivated in this process. DNA for integration can be assembled by PCR amplifications of synthetic DNA consisting of the following DNA components: amyE 5′ region+synthetic consensus promoter with SD sequence (PconsSD)+aprH open reading frame+the B. amyloliquefaciens amyQ terminator+the cat gene (conferring resistance to chloramphenicol)+amyE 3′ region. The purified PCR products were used in a subsequent PCR reaction to create a single linear DNA by the SOE method described in Example 2. The resulting SOE product was used directly for transformation of the B. subtilis strains AN2 (resulting in strain AQG88), AN2406 (resulting in strain AQG825), and AN2407 (resulting in strain AQG812). Chromosomal integration was facilitated by homologous recombination and cells wherein double cross-over events occurred were selected for on LB agar plates containing 6 μg/ml chloramphenicol. The strain AQG88 expresses B. clausii AprH (SEQ ID NO: 3) from the amyE locus and contains the native pel locus. The strain AQG825 expresses PrsA from Bacillus sp. (SEQ ID NO: 6) from the pel locus and AprH from the amyE locus. The strain AQG812 expresses PrsA from Geobacillus caldoxylosilyticus (SEQ ID NO: 9) from the pel locus and AprH from the amyE locus. A schematic view of the linear DNA product used for integration of the aprH gene in B. subtilis strains AN2, AN2406, and AN2407 is shown in FIG. 3 and the DNA sequence is provided as SEQ ID NO: 14.

### Example 4. Protease Expression in Batch Cultures with B. subtilis AQG88, AQG825 and AQG812

The B. subtilis strains constructed in Example 3 were tested with respect to protease productivity in standard microplate batch-cultivations as described above. Cultivations were carried out in biological triplicates for 24 hours, after which the supernatant was harvested for subsequent protease activity measurements as described above. In this example we obtained 14% more protease activity from cultures with AQG825 expressing the Bacillus sp. PrsA (SEQ ID NO: 6) and 23% more protease activity from cultures with AQG812 expressing the Geobacillus caldoxylosilyticus PrsA (SEQ ID NO: 9) as compared to cultures with AQG88 that does not express any heterologous prsA genes.

1-17. (canceled)

18. A gram-positive host cell comprising in its genome:
a) a first heterologous promoter operably linked to at least one polynucleotide encoding a protease; and
b) a second heterologous promoter operably linked to at least one polynucleotide encoding a foldase;
wherein the foldase has a sequence identity of at least 80% to SEQ ID NO: 6 or SEQ ID NO: 9.

19. The host cell according to claim 18, wherein the first heterologous promoter and the second heterologous promoter are different.

20. The host cell according to claim 18, wherein the first heterologous promoter and the second heterologous promoter are identical copies of the same promoter.

21. The host cell according to claim 18, wherein the protease is a serine protease, cysteine protease, threonine protease, aspartic protease, glutamic protease, metalloprotease, or asparagine peptide lyase.

22. The host cell according to claim 21, wherein the protease is a serine protease.

23. The host cell according to claim 21, wherein the protease is a subtilase.

24. The host cell according to claim 21, wherein the protease is a subtilisin.

25. The host cell according to claim 18, wherein the protease comprises a C- or N-terminal propeptide and/or an N-terminal signal peptide.

26. The host cell according to claim 18, wherein the protease wherein is a mature protease.

27. The host cell according to claim 18, wherein the protease has a sequence identity of at least 80% to SEQ ID NO: 3.

28. The host cell according to claim 18, wherein the protease comprises or consists of SEQ ID NO: 3.

29. The host cell according to claim 18, wherein the protease is a Bacillus clausii alkaline protease (AprH) or a variant thereof.

30. The host cell according to claim 18, wherein the at least one polynucleotide encoding a protease has a sequence identity of at least 80% to the mature polypeptide coding sequence of SEQ ID NO: 1.

31. The host cell according to claim 18, wherein the at least one polynucleotide encoding a protease comprises or consists of SEQ ID NO: 1.

32. The host cell according to claim 18, wherein the at least one polynucleotide encoding a foldase has a sequence identity of at least 80% to the mature polypeptide coding sequence of SEQ ID NO: 4 or SEQ ID NO: 7.

33. The host cell according to claim 18, wherein the at least one polynucleotide encoding a foldase comprises or consists of SEQ ID NO: 4 or SEQ ID NO: 7.

34. The Gram-positive host cell according to claim 18, wherein the Gram-positive host cell is a Bacillus host cell

35. The Gram-positive host cell according to claim 34, wherein the Gram-positive host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cell.

36. A method for producing a protease, the method comprising:
a) providing a Gram-positive host cell according to claim 18;
b) cultivating said Gram-positive host cell under conditions conducive for expression of the protease and the foldase.
and, optionally

37. A method for producing a protease of claim 36, wherein the method further comprises: c) recovering the protease.


SEQUENCE LISTING (14 sequences)

SEQ ID NO: 1 - DNA, 1140 bp, Bacillus clausii
  CDS (1)..(1140)
  sig_peptide (1)..(81)
  mat_peptide (82)..(1140)
atgaagaaaccgttggggaaaattgtcgcaagcaccgcactactcatttctgttgcttttagttcatcgatcgcatcggctgctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcagtgccatggggaattagccgtgtgcaagccccagctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggccgggacgattgctgctttaaacaattcgattggcgttcttggcgtagcgccgagcgcggaactatacgctgttaaagtattaggggcgagcggttcaggttcggtcagctcgattgcccaaggattggaatgggcagggaacaatggcatgcacgttgctaatttgagtttaggaagcccttcgccaagtgccacacttgagcaagctgttaatagcgcgacttctagaggcgttcttgttgtagcggcatctgggaattcaggtgcaggctcaatcagctatccggcccgttatgcgaacgcaatggcagtcggagctactgaccaaaacaacaaccgcgccagcttttcacagtatggcgcagggcttgacattgtcgcaccaggtgtaaacgtgcagagcacatacccaggttcaacgtatgccagcttaaacggtacatcgatggctactcctcatgttgcaggtgcagcagcccttgttaaacaaaagaacccatcttggtccaatgtacaaatccgcaatcatctaaagaatacggcaacgagcttaggaagcacgaacttgtatggaagcggacttgtcaatgcagaagcggcaacacgc

SEQ ID NO: 2 - protein, 380 aa, Bacillus clausii
MKKPLGKIVASTALLISVAFSSSIASAAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 353 aa, Bacillus clausii
AEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 4 - DNA, 879 bp, Artificial Sequence
  Polynucleotide sequence encoding PrsA consisting of the signal peptide of B. subtilis PrsA and the mature polypeptide of Bacillus sp. PrsA.
  CDS (1)..(876)
  sig_peptide (1)..(57)
  mat_peptide (58)..(876)
atgaagaaaatcgcaatagcagctatcactgctacaagcatcctcgctctcagtgcttgcggcaacgctggcaacgagaaggttgcaacatcaaaggttggcgacgtaacgaaggaccaactttacaacgagatgaaggactctgttggcgactctgcgcttcaacttatcatcatcgagaaggtacttaacgacaagtacaaggtttcagacaaagaggtagaggcagagttcaagaagcaaaaggagcagatgggcagctcttacgagcaaactcttgctgcacagaacatgactgagaagtctttcaaacgtggcatcaagcttaaccttcttcaggaagccgctcttgcggacggcgttaaggtttctgacgctgagatgaaggcatactacgaggagatgaagaaagaggtaaaggcttctcatatccttgtagctgacgagaagactgctaaggagatcaaggctaagttagacaagggcgaggacttcgcaactcttgctaagaagtactctacggacacaggctctcaggcagcaggtggcgagcttggctggtttggccctgacaagatggttcctgagttcactaaggctgcttactctcttaagaagggcgagatcagcgagcctgtaaagacttcttacggctaccatatcatcaaggtagaggacatccgcgacgctaaggtaaagggcacgtacgaggacaacaaggctgctatcaagaagaagcttcaacttcagaaggcagaccaatcacaacttctttcaaaggtttcaaagcttatcaaggacgcggacgttaagatcgaggacaaggaccttaaggacgctcttgcacaattcacgggcagcacagcagctccaaagtaa

SEQ ID NO: 5 - protein, 292 aa, Artificial Sequence
  Synthetic Construct
MKKIAIAAITATSILALSACGNAGNEKVATSKVGDVTKDQLYNEMKDSVGDSALQLIIIEKVLNDKYKVSDKEVEAEFKKQKEQMGSSYEQTLAAQNMTEKSFKRGIKLNLLQEAALADGVKVSDAEMKAYYEEMKKEVKASHILVADEKTAKEIKAKLDKGEDFATLAKKYSTDTGSQAAGGELGWFGPDKMVPEFTKAAYSLKKGEISEPVKTSYGYHIIKVEDIRDAKVKGTYEDNKAAIKKKLQLQKADQSQLLSKVSKLIKDADVKIEDKDLKDALAQFTGSTAAPK

SEQ ID NO: 6 - protein, 273 aa, Bacillus sp.
CGNAGNEKVATSKVGDVTKDQLYNEMKDSVGDSALQLIIIEKVLNDKYKVSDKEVEAEFKKQKEQMGSSYEQTLAAQNMTEKSFKRGIKLNLLQEAALADGVKVSDAEMKAYYEEMKKEVKASHILVADEKTAKEIKAKLDKGEDFATLAKKYSTDTGSQAAGGELGWFGPDKMVPEFTKAAYSLKKGEISEPVKTSYGYHIIKVEDIRDAKVKGTYEDNKAAIKKKLQLQKADQSQLLSKVSKLIKDADVKIEDKDLKDALAQFTGSTAAPK

SEQ ID NO: 7 - DNA, 834 bp, Artificial Sequence
  Polynucleotide sequence encoding PrsA consisting of the signal peptide of B. subtilis PrsA and the mature polypeptide of Geobacillus caldoxylosilyticus PrsA
  CDS (1)..(831)
  sig_peptide (1)..(57)
  mat_peptide (58)..(831)
atgaagaaaatcgcaatagcagctatcactgctacaagcatcctcgctctcagtgcttgcaacaatggtggctcagaggttatcgtaaagactaaggacggcaacatcactaaggaggagttctacaacgagatgaaagctcgcgttggcaaagaggttatccgcgaccttatcgacgagaaggtactttcaaagaagtacaaggttacagacaaggagatcgacaagcaaatcgagaaccttaaggaggcgtacggcacgcagtacgaccttgcagtacaacagaacggcgagaaggctatccgcgagatggttaagcttgaccttcttcgccaaaaagctgccatggaggatattaaagtcaccgaaaaagaacttaaggagtactacaactcatacaagcctaagattcgcgcatcacatatccttgttaaggacgagaagacggcgaaggagatcaaggctaagttagacaagggcgaggacttcgcgaagcttgcaaagcaatactcacaggaccctggctcagctgcgaatggtggcgaccttggctggtttggccctggcaagatggttaaggagttcgaggacgctgcgtacaagcttaaggtaggccaagtttcagacccggttaagacagactacggctaccatatcatcaaggttactgctaaggagaagaagaagccattcaacgagatgaaggacgagatcgagttcgaggtaaagcaacgcaagcttgaccctacgaaggttcaatcaaaggtagagaagcttgtaaaggacgctaaggttgagatcgaggacaaggaccttcaagacgttcttaagtaa

SEQ ID NO: 8 - protein, 277 aa, Artificial Sequence
  Synthetic Construct
MKKIAIAAITATSILALSACNNGGSEVIVKTKDGNITKEEFYNEMKARVGKEVIRDLIDEKVLSKKYKVTDKEIDKQIENLKEAYGTQYDLAVQQNGEKAIREMVKLDLLRQKAAMEDIKVTEKELKEYYNSYKPKIRASHILVKDEKTAKEIKAKLDKGEDFAKLAKQYSQDPGSAANGGDLGWFGPGKMVKEFEDAAYKLKVGQVSDPVKTDYGYHIIKVTAKEKKKPFNEMKDEIEFEVKQRKLDPTKVQSKVEKLVKDAKVEIEDKDLQDVLK

SEQ ID NO: 9 - protein, 258 aa, Geobacillus caldoxylosilyticus
CNNGGSEVIVKTKDGNITKEEFYNEMKARVGKEVIRDLIDEKVLSKKYKVTDKEIDKQIENLKEAYGTQYDLAVQQNGEKAIREMVKLDLLRQKAAMEDIKVTEKELKEYYNSYKPKIRASHILVKDEKTAKEIKAKLDKGEDFAKLAKQYSQDPGSAANGGDLGWFGPGKMVKEFEDAAYKLKVGQVSDPVKTDYGYHIIKVTAKEKKKPFNEMKDEIEFEVKQRKLDPTKVQSKVEKLVKDAKVEIEDKDLQDVLK

SEQ ID NO: 10 - DNA, 768 bp, Artificial Sequence
  sigF
atggatgtggaggttaagaaaaacggcaaaaacgctcagctgaaggatcatgaagtaaaggaattaatcaaacaaagccaaaatggcgaccagcaggcaagagacctcctcatagaaaaaaacatgcgtcttgtttggtctgtcgtacagcggtttttaaacagaggatatgagcctgacgatctcttccagatcggctgcatcgggctgttaaaatctgttgacaaatttgatttaacctatgatgtgcgtttttcaacgtatgcagtgccgatgattatcggagaaatccaacgatttatccgtgatgacggaaccgtaaaggtatcacggtcattaaaagagcttggaaacaaaatccggcgcgcgaaggatgagctttcgaaaacactgggcagagtgccgacggtgcaggagatcgctgaccatttggagattgaagctgaggatgttgtactggcccaagaggcggtaagggctccatcttcgattcacgaaaccgtttatgaaaatgacggagatccgattaccctgcttgatcaaatcgctgacaactcagaagaaaaatggtttgacaaaattgcgctgaaagaagcgatcagcgatttggaggaaagggaaaaactaatcgtctatctcagatattataaagaccagacacagtccgaggtggctgagcggctcgggatctctcaggtgcaggtttccaggcttgaaaagaaaatattaaaacagatcaaggttcaaatggatcatacggatggctag

SEQ ID NO: 11 - DNA, 471 bp, Artificial Sequence
  sigF delta 297 bp
atggatgtggaggttaagaaaaacggcaaaaacgctcagctgaaggatcatgaagtaaaggaattaatcaaacaaagccaaaatggcgaccagcaggcaagagacctcctcatagaaaaaaacatgcgtcttgtttggtctgtcgtacagcggtttttaaacagaggatatgagcctgacgatctcttccagatcggctgcatcgggctggaaaatgacggagatccgattaccctgcttgatcaaatcgctgacaactcagaagaaaaatggtttgacaaaattgcgctgaaagaagcgatcagcgatttggaggaaagggaaaaactaatcgtctatctcagatattataaagaccagacacagtccgaggtggctgagcggctcgggatctctcaggtgcaggtttccaggcttgaaaagaaaatattaaaacagatcaaggttcaaatggatcatacggatggctag

SEQ ID NO: 12 - DNA, 8135 bp, Artificial Sequence
  SOE PCR product for integration of the prsA gene from Bacillus sp. in AN2
gtctcacttccttactgcgtctggttgcaaaaacgaagaagcaaggattcccctcgcttctcatttgtcctatttattatacacttttttaggcacatctttggcgcttgtttcactagacttgatgcctctgaatcttgtccaagtgtcacggtccgcatcatagacttgtccatttttcaccgctttgagatttttccagagcgggttcgttttccactcatctacaatggttttgccttcgttggctgagatgaacaaaatatcaggatcgattttgctcaattgctcaaggctgacctcttgataggcgttatctgacttcacagcgtgtgtaaagcctagcattttaaagatttctccgtcataggatgatgatgtatgaagctggaaggaatccgctcttgcaacgccgagaacgatgttgcggttttcatctttcggaagttcggcttttagatcgttgatgacttttatgtgctcggcaagcttttcttttccttcatcttctttatttaatgctttagcaatggtcgtaaagctgtcgatcgtttcgtcatatgtcgcttcacggctttttaattcaatcgtcggggcgatttttttcagctgtttataaatgtttttatggcgctcagcgtcagcgatgattaaatcaggcttcaaggaactgatgacctcaagattgggttcgctgcgtgtgcctacagatgtgtaatcaatggagctgccgacaagctttttaatcatatcttttttgttgtcatctgcgatgcccaccggcgtaatgccgagattgtgaacggcatccaagaatgaaagctcaagcacaaccacccgctaaggtgtgccgcttactgtcgtttttccttcttcgtcatggatcactctggaatccttagactcgcttttgccgcttccgttgttattctggcttgatgaacagccggatacaatgaggcaggcgagcaataaaacactcatgatggcaatcaacttgttagaataggtgcgcatgtcattcttccttttttcagatttagtaatgagaatcattatcacatgtaacactataatagcatggcttatcatgtcaatatttttttagtaaagaaagctgcgtttttactgctttctcatgaaagcatcatcagacacaaataagtggtatgcagcgttaccgtgtcttcgagacaaaaacgcatgggcgttggctttagaggtttcgaacatatcagcagtgacataaggaaggagagtgctgagataaccggacaatttcttttctatttcatctgttagtgcaaattcaatgtcgccgatattcatgataatcgagaaaacaaagtcgatatcgatatgaaaatgttcctcggcaaaaaccgcaagctcgtgaattcctggtgaacatccggcacgcttatggaaaatctgtttgactaaatcactcacaatccaagcattgtattgctgttctggtgaaaagtattgcattagacatacctcctgctcgtacggataaaggcagcgtttcatggtcgtgtgctccgtgcagcggcttctccttaattttgatttttctgaaaataggtcccgttcctatcactttaccatggacggaaaacaaatagctactaccattcctcctgtttttctcttcaatgttctggaatctgtttcaggtacagacgatcgggtatgaaagaaatatagaaaacatgaaggaggaatatcgacatgaaaccagttgtaaaagagtatacaaatgacgaacagctcatgaaagatgtagaggaattgcagaaaatgggtgttgcgaaagaggatgtatacgtcttagctcacgacgatgacagaacggaacgcctggctgacaacacgaacgccaacacgatcggagccaaagaaacaggttttaagcacgcggtgggaaatatcttcaataaaaaaggagacgagctccgcaataaaattcacgaaatcggtttttctgaagatgaagccgctcaatttgaaaaacgcttagatgaaggaaaagtgcttctctttgtgacagataacgaaaaagtgaaagcttgggcataaagcaaggaaaaaaccaaaaggccaatgtcggccttttggtttttttgcggtctttgcggtgggattttgcagaatgccgcaataggatagcggaacattttcggttctgaatgtccctcaatttgctattatatttttgtgataaattggaataaaatctcacaaaatagaaaatgggggtacatagtggatgaaaaaagtgatgttagctacggctttgtttttaggattgactccagctggcgcgaacgcagctgatttaggccaccagacgttgggatccaatgatggctggggcgcgtactcgaccggcacgacaggcggatcaaaagcaccctcctcaaatgtgtataccgtcagcaacagaaaccagcttgtctcggcattagggaaagaaacgaacacaacgccaaaaatcatttatatcaagggaacgattgacatgaacgtggatgacaatctgaagccgcttggcctaaatgactataaagatccggagtatgatttggacaaatatttgaaagcctatgatcctagcacatggggcaaaaaagagccgtcgggaacacaagaagaagcgagagcacgctctcagaaaaaccaaaaagcacgggtcatggtggatatccctgcaaacacgacgatcgtcggttcagggactaacgctaaagtcgtgggaggaaacttccaaatcaagagtgataacgtcattattcgcaacattgaattccaggatgcctatgactattttccgcaatggttgtaaaacgacggccagtgaattctgatcaaatggttcagtgagagcgaagcgaacacttgattttttaattttctatcttttataggtcattagagtatacttatttgtcctataaactatttagcagcataatagatttattgaataggtcatttaagttgagcgtattagaggaggaaaatcttggagaaatatttgaagaacccgaacgcgtataataaagaataataataaatctgtagacaaattgtgaaaggatgtacttaaacgctaacggtcagctttattgaacagtaatttaagtatatgtccaatctagggtaagtaaattgagtatcaatataaactttatatgaacataatcaacgaggtgaaatcatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaggctaattttattgcaataacaggtgcttacttttaaaactactgatttattgataaatattgaacaatttttgggaagaataaagcgtcctcttgtgaaattagagaacgctttattactttaatttagtgaaacaatttgtaactattgaaaatagaaagaaattgttccttcgatagtttattaatattagtggagctcagtgagagcgaagcgaacacttgattttttaattttctatcttttataggtcattagagtatacttatttgtcctataaactatttagcagcataatagatttattgaataggtcatttaagttgagcatattaggggaggaaaatcttggagaaatatttgaagaacccgagatctagatcaggtacctcaggatgattgatcacccgcggtgtaaaaaataggaataaaggggggttgacattattttactgatatgtataatataatttgtataagaaaatgagagggagaggaaacatgaagaaaatcgcaatagcagctatcactgctacaagcatcctcgctctcagtgcttgcggcaacgctggcaacgagaaggttgcaacatcaaaggttggcgacgtaacgaaggaccaactttacaacgagatgaaggactctgttggcgactctgcgcttcaacttatcatcatcgagaaggtacttaacgacaagtacaaggtttcagacaaagaggtagaggcagagttcaagaagcaaaaggagcagatgggcagctcttacgagcaaactcttgctgcacagaacatgactgagaagtctttcaaacgtggcatcaagcttaaccttcttcaggaagccgctcttgcggacggcgttaaggtttctgacgctgagatgaaggcatactacgaggagatgaagaaagaggtaaaggcttctcatatccttgtagctgacgagaagactgctaaggagatcaaggctaagttagacaagggcgaggacttcgcaactcttgctaagaagtactctacggacacaggctctcaggcagcaggtggcgagcttggctggtttggccctgacaagatggttcctgagttcactaaggctgcttactctcttaagaagggcgagatcagcgagcctgtaaagacttcttacggctaccatatcatcaaggtagaggacatccgcgacgctaaggtaaagggcacgtacgaggacaacaaggctgctatcaagaagaagcttcaacttcagaaggcagaccaatcacaacttctttcaaaggtttcaaagcttatcaaggacgcggacgttaagatcgaggacaaggaccttaaggacgctcttgcacaattcacgggcagcacagcagctccaaagtaaggacgccgtctctgcatggatcgattgatgcttctgctaatgtgaaatcaaatgttataaatcaagcgggtgcgggtaaattaaattaagaaagtgaaaaacacaaagggtgctaacctttgtgttttttaattaattaaaatgtttattaacttagttaaggagtagaatggaaaaggggatcggaaaacaagtatataggaggagacctatttatggcttcagaaaaagacgcaggaaaacagtcagcagtaaagcttgttccattgcttattactgtcgctgtgggactaatcatctggtttattcccgctccgtccggacttgaacctaaagcttggcatttgtttgcgatttttgtcgcaacaattatcggctttatctccaagcccttgccaatgggtgcaattgcaatttttgcattggcggttactgcactaactggaacactatcaattgaggatacattaagcggattcgggaataagaccatttggcttatcgttatcgcattctttatttcccggggatttatcaaaaccggtctcggtgcgagaatttcgtatgtattcgttcagaaattcggaaaaaaaacccttggactttcttattcactgctattcagtgatttaatactttcacctgctattccaagtaatacggcgcgtgcaggaggcattatatttcctattatcagatcattatccgaaacattcggatcaagcccggcaaatggaacagagagaaaaatcggtgcattcttattaaaaaccggttttcaggggaatctgatcacatctgctatgttcctgacagcgatggcggcgaacccgctgattgccaagctggcccatgatgtcgcaggggtggacttaacatggacaagctgggcaattgccgcgattgtaccgggacttgtaagcttaatcatcacgccgcttgtgatttacaaactgtatccgccggaaatcaaagaaacaccggatgcggcgaaaatcgcaacagaaaaactgaaagaaatgggaccgttcaaaaaatcggagctttccatggttatcgtgtttcttttggtgcttgtgctgtggatttttggcggcagcttcaacatcgacgctaccacaaccgcattgatcggtttggccgttctcttattatcacaagttctgacttgggatgatatcaagaaagaacagggcgcttgggatacgctcacttggtttgcggcgcttgtcatgctcgccaacttcttgaatgaattaggcatggtgtcttggttcagtaatgccatgaaatcatccgtatcagggttctcttggattgtggcattcatcattttaattgttgtgtattattactctcactatttctttgcaagtgcgacagcccacatcagtgcgatgtattcagcatttttggctgtcgtcgtggcagcgggcgcaccgccgcttttagcagcgctgagcctcgcgttcatcagcaacctgttcgggtcaacgactcactacggttctggagcggctccggtcttcttcggagcaggctacatcccgcaaggcaaatggtggtccatcggatttatcctgtcgattgttcatatcatcgtatggcttgtgatcggcggattatggtggaaagtactaggaatatggtagaaagaaaaaggcagacgcggtctgcctttttttattttcactccttcgtaagaaaatggattttgaaaaatgagaaaattccctgtgaaaaatggtatgatctaggtagaaaggacggctggtgctgtggtgaaaaagcggttccatttttccctgcaaacaaaaataatggggctgattgcggctctgctggtctttgtcattggtgtgctgaccattacgttagccgttcagcatacacagggagaacggagacaggcagagcagctggcggttcaaacggcgagaaccatttcctatatgccgccggttaaagagctcattgagagaaaagacggacatgcggctcagacgcaagaggtcattgaacaaatgaaagaacagactggtgcgtttgccatttatgttttgaacgaaaaaggagacattcgcagcgcctctggaaaaagcggattaaagaaactggagcgcagcagagaaattttgtttggcggttcgcatgtttctgaaacaaaagcggatggacgaagagtgatcagagggagcgcgccgattataaaagaacagaagggatacagccaagtgatcggcagcgtgtctgttgattttctgcaaacggagacagagcaaagcatcaaaaagcatttgagaaatttgagtgtgattgctgtgcttgtactgctgctcggatttattggcgccgccgtgctggcgaaaagcatcagaaaggatacgctcgggcttgaaccgcatgagatcgcggctctatatcgtgagaggaacgcaatgcttttcgcgattcgagaagggattattgccaccaatcgtgaaggcgtcgtcaccatgatgaacgtatcggcggccgagatgctgaagctgcccgagcctgtgatccatcttcctatagatgacgtcatgccgggagcagggctgatgtctgtgcttgaaaaaggagaaatgctgccgaaccaggaagtaagcgtcaacgatcaagtgtttattatcaatacgaaagtgatgaatcaaggcgggcaggcgtatgggattgtcgtcagcttcagggagaaaacagagctgaagaagctgatcgacacattgacagaggttcgcaaatattcagaggatctcagggcgcagactca

SEQ ID NO: 13 - DNA, 8089 bp, Artificial Sequence
  SOE PCR product for integration of the prsA gene from Geobacillus caldoxylosilyticus in AN2
gtctcacttccttactgcgtctggttgcaaaaacgaagaagcaaggattcccctcgcttctcatttgtcctatttattatacacttttttaggcacatctttggcgcttgtttcactagacttgatgcctctgaatcttgtccaagtgtcacggtccgcatcatagacttgtccatttttcaccgctttgagatttttccagagcgggttcgttttccactcatctacaatggttttgccttcgttggctgagatgaacaaaatatcaggatcgattttgctcaattgctcaaggctgacctcttgataggcgttatctgacttcacagcgtgtgtaaagcctagcattttaaagatttctccgtcataggatgatgatgtatgaagctggaaggaatccgctcttgcaacgccgagaacgatgttgcggttttcatctttcggaagttcggcttttagatcgttgatgacttttatgtgctcggcaagcttttcttttccttcatcttctttatttaatgctttagcaatggtcgtaaagctgtcgatcgtttcgtcatatgtcgcttcacggctttttaattcaatcgtcggggcgatttttttcagctgtttataaatgtttttatggcgctcagcgtcagcgatgattaaatcaggcttcaaggaactgatgacctcaagattgggttcgctgcgtgtgcctacagatgtgtaatcaatggagctgccgacaagctttttaatcatatcttttttgttgtcatctgcgatgcccaccggcgtaatgccgagattgtgaacggcatccaagaatgaaagctcaagcacaaccacccgctaaggtgtgccgcttactgtcgtttttccttcttcgtcatggatcactctggaatccttagactcgcttttgccgcttccgttgttattctggcttgatgaacagccggatacaatgaggcaggcgagcaataaaacactcatgatggcaatcaacttgttagaataggtgcgcatgtcattcttccttttttcagatttagtaatgagaatcattatcacatgtaacactataatagcatggcttatcatgtcaatatttttttagtaaagaaagctgcgtttttactgctttctcatgaaagcatcatcagacacaaataagtggtatgcagcgttaccgtgtcttcgagacaaaaacgcatgggcgttggctttagaggtttcgaacatatcagcagtgacataaggaaggagagtgctgagataaccggacaatttcttttctatttcatctgttagtgcaaattcaatgtcgccgatattcatgataatcgagaaaacaaagtcgatatcgatatgaaaatgttcctcggcaaaaaccgcaagctcgtgaattcctggtgaacatccggcacgcttatggaaaatctgtttgactaaatcactcacaatccaagcattgtattgctgttctggtgaaaagtattgcattagacatacctcctgctcgtacggataaaggcagcgtttcatggtcgtgtgctccgtgcagcggcttctccttaattttgatttttctgaaaataggtcccgttcctatcactttaccatggacggaaaacaaatagctactaccattcctcctgtttttctcttcaatgttctggaatctgtttcaggtacagacgatcgggtatgaaagaaatatagaaaacatgaaggaggaatatcgacatgaaaccagttgtaaaagagtatacaaatgacgaacagctcatgaaagatgtagaggaattgcagaaaatgggtgttgcgaaagaggatgtatacgtcttagctcacgacgatgacagaacggaacgcctggctgacaacacgaacgccaacacgatcggagccaaagaaacaggttttaagcacgcggtgggaaatatcttcaataaaaaaggagacgagctccgcaataaaattcacgaaatcggtttttctgaagatgaagccgctcaatttgaaaaacgcttagatgaaggaaaagtgcttctctttgtgacagataacgaaaaagtgaaagcttgggcataaagcaaggaaaaaaccaaaaggccaatgtcggccttttggtttttttgcggtctttgcggtgggattttgcagaatgccgcaataggatagcggaacattttcggttctgaatgtccctcaatttgctattatatttttgtgataaattggaataaaatctcacaaaatagaaaatgggggtacatagtggatgaaaaaagtgatgttagctacggctttgtttttaggattgactccagctggcgcgaacgcagctgatttaggccaccagacgttgggatccaatgatggctggggcgcgtactcgaccggcacgacaggcggatcaaaagcaccctcctcaaatgtgtataccgtcagcaacagaaaccagcttgtctcggcattagggaaagaaacgaacacaacgccaaaaatcatttatatcaagggaacgattgacatgaacgtggatgacaatctgaagccgcttggcctaaatgactataaagatccggagtatgatttggacaaatatttgaaagcctatgatcctagcacatggggcaaaaaagagccgtcgggaacacaagaagaagcgagagcacgctctcagaaaaaccaaaaagcacgggtcatggtggatatccctgcaaacacgacgatcgtcggttcagggactaacgctaaagtcgtgggaggaaacttccaaatcaagagtgataacgtcattattcgcaacattgaattccaggatgcctatgactattttccgcaatggttgtaaaacgacggccagtgaattctgatcaaatggttcagtgagagcgaagcgaacacttgattttttaattttctatcttttataggtcattagagtatacttatttgtcctataaactatttagcagcataatagatttattgaataggtcatttaagttgagcgtattagaggaggaaaatcttggagaaatatttgaagaacccgaacgcgtataataaagaataataataaatctgtagacaaattgtgaaaggatgtacttaaacgctaacggtcagctttattgaacagtaatttaagtatatgtccaatctagggtaagtaaattgagtatcaatataaactttatatgaacataatcaacgaggtgaaatcatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaggctaattttattgcaataacaggtgcttacttttaaaactactgatttattgataaatattgaacaatttttgggaagaataaagcgtcctcttgtgaaattagagaacgctttattactttaatttagtgaaacaatttgtaactattgaaaatagaaagaaattgttccttcgatagtttattaatattagtggagctcagtgagagcgaagcgaacacttgattttttaattttctatcttttataggtcattagagtatacttatttgtcctataaactatttagcagcataatagatttattgaataggtcatttaagttgagcatattaggggaggaaaatcttggagaaatatttgaagaacccgagatctagatcaggtacctcaggatgattgatcacccgcggtgtaaaaaataggaataaaggggggttgacattattttactgatatgtataatataatttgtataagaaaatgagagggagaggaaacatgaagaaaatcgcaatagcagctatcactgctacaagcatcctcgctctcagtgcttgcaacaatggtggctcagaggttatcgtaaagactaaggacggcaacatcactaaggaggagttctacaacgagatgaaagctcgcgttggcaaagaggttatccgcgaccttatcgacgagaaggtactttcaaagaagtacaaggttacagacaaggagatcgacaagcaaatcgagaaccttaaggaggcgtacggcacgcagtacgaccttgcagtacaacagaacggcgagaaggctatccgcgagatggttaagcttgaccttcttcgccaaaaagctgccatggaggatattaaagtcaccgaaaaagaacttaaggagtactacaactcatacaagcctaagattcgcgcatcacatatccttgttaaggacgagaagacggcgaaggagatcaaggctaagttagacaagggcgaggacttcgcgaagcttgcaaagcaatactcacaggaccctggctcagctgcgaatggtggcgaccttggctggtttggccctggcaagatggttaaggagttcgaggacgctgcgtacaagcttaaggtaggccaagtttcagacccggttaagacagactacggctaccatatcatcaaggttactgctaaggagaagaagaagccattcaacgagatgaaggacgagatcgagttcgaggtaaagcaacgcaagcttgaccctacgaaggttcaatcaaaggtagagaagcttgtaaaggacgctaaggttgagatcgaggacaaggaccttcaagacgttcttaagtaaggacgccgtctctgcatggatcgattgatgcttctgctaatgtgaaatcaaatgttataaatcaagcgggtgcgggtaaattaaattaagaaagtgaaaaacacaaagggtgctaacctttgtgttttttaattaattaaaatgtttattaacttagttaaggagtagaatggaaaaggggatcggaaaacaagtatataggaggagacctatttatggcttcagaaaaagacgcaggaaaacagtcagcagtaaagcttgttccattgcttattactgtcgctgtgggactaatcatctggtttattcccgctccgtccggacttgaacctaaagcttggcatttgtttgcgatttttgtcgcaacaattatcggctttatctccaagcccttgccaatgggtgcaattgcaatttttgcattggcggttactgcactaactggaacactatcaattgaggatacattaagcggattcgggaataagaccatttggcttatcgttatcgcattctttatttcccggggatttatcaaaaccggtctcggtgcgagaatttcgtatgtattcgttcagaaattcggaaaaaaaacccttggactttcttattcactgctattcagtgatttaatactttcacctgctattccaagtaatacggcgcgtgcaggaggcattatatttcctattatcagatcattatccgaaacattcggatcaagcccggcaaatggaacagagagaaaaatcggtgcattcttattaaaaaccggttttcaggggaatctgatcacatctgctatgttcctgacagcgatggcggcgaacccgctgattgccaagctggcccatgatgtcgcaggggtggacttaacatggacaagctgggcaattgccgcgattgtaccgggacttgtaagcttaatcatcacgccgcttgtgatttacaaactgtatccgccggaaatcaaagaaacaccggatgcggcgaaaatcgcaacagaaaaactgaaagaaatgggaccgttcaaaaaatcggagctttccatggttatcgtgtttcttttggtgcttgtgctgtggatttttggcggcagcttcaacatcgacgctaccacaaccgcattgatcggtttggccgttctcttattatcacaagttctgacttgggatgatatcaagaaagaacagggcgcttgggatacgctcacttggtttgcggcgcttgtcatgctcgccaacttcttgaatgaattaggcatggtgtcttggttcagtaatgccatgaaatcatccgtatcagggttctcttggattgtggcattcatcattttaattgttgtgtattattactctcactatttctttgcaagtgcgacagcccacatcagtgcgatgtattcagcatttttggctgtcgtcgtggcagcgggcgcaccgccgcttttagcagcgctgagcctcgcgttcatcagcaacctgttcgggtcaacgactcactacggttctggagcggctccggtcttcttcggagcaggctacatcccgcaaggcaaatggtggtccatcggatttatcctgtcgattgttcatatcatcgtatggcttgtgatcggcggattatggtggaaagtactaggaatatggtagaaagaaaaaggcagacgcggtctgcctttttttattttcactccttcgtaagaaaatggattttgaaaaatgagaaaattccctgtgaaaaatggtatgatctaggtagaaaggacggctggtgctgtggtgaaaaagcggttccatttttccctgcaaacaaaaataatggggctgattgcggctctgctggtctttgtcattggtgtgctgaccattacgttagccgttcagcatacacagggagaacggagacaggcagagcagctggcggttcaaacggcgagaaccatttcctatatgccgccggttaaagagctcattgagagaaaagacggacatgcggctcagacgcaagaggtcattgaacaaatgaaagaacagactggtgcgtttgccatttatgttttgaacgaaaaaggagacattcgcagcgcctctggaaaaagcggattaaagaaactggagcgcagcagagaaattttgtttggcggttcgcatgtttctgaaacaaaagcggatggacgaagagtgatcagagggagcgcgccgattataaaagaacagaagggatacagccaagtgatcggcagcgtgtctgttgattttctgcaaacggagacagagcaaagcatcaaaaagcatttgagaaatttgagtgtgattgctgtgcttgtactgctgctcggatttattggcgccgccgtgctggcgaaaagcatcagaaaggatacgctcgggcttgaaccgcatgagatcgcggctctatatcgtgagaggaacgcaatgcttttcgcgattcgagaagggattattgccaccaatcgtgaaggcgtcgtcaccatgatgaacgtatcggcggccgagatgctgaagctgcccgagcctgtgatccatcttcctatagatgacgtcatgccgggagcagggctgatgtctgtgcttgaaaaaggagaaatgctgccgaaccaggaagtaagcgtcaacgatcaagtgtttattatcaatacgaaagtgatgaatcaaggcgggcaggcgtatgggattgtcgtcagcttcagggagaaaacagagctgaagaagctgatcgacacattgacagaggttcgcaaatattcagaggatctcagggcgcagactc

SEQ ID NO: 14 - DNA, 9119 bp, Artificial Sequence
  SOE PCR product for integration of the aprH gene from Bacillus clausii in AN2, AN2406, and AN2407
gcattaacgtgcccaatgccattgtcatatgtgaatcgtgtccgcaggaatggttggcgcgaaatgtgccgttaacctcctgccacagcgcgtcaatatcagcgcgtaccgctacaacaggtgagcctgagccgatttcgccgacaaccccggtgcagtctgaaaacgtgcgcgtccggcaccctaaatcctcaagcttttgtttcaaaaatgaagttgtctcatattccttccagctgacttcagggttcgcgtgcagatgctcgaagatgtccataatggtttgtttcatttcttctgaaagcttttgcatggtaagaaatacctccttctatcagaatgaatttttaccttctttactttatttatattgaaacaggaagataggctgtatataatatagcacatattgctactattcagaataattaatattttcaaacagaggggatggatcgaaatatgagtatgccagcagccgaaacacagcctaagaaaaaacgtatgacatttaaaatgcctgacgcctatgtcctcttatttatgattgctttcatttgcgcaatcgcttcatatattgtgccggcaggtgaatttgaccgcgtgacaaagggggatgtcacgaccgctgttccgggaagctatcattcaattgaacagtctccggtcagattgatcagcttttttacttctctacaggatggaatggttggatcagcacccatcatctttctgattttattcacaggcggcaccattgctattctagaaaaaacgggtgccatcaatggcctgatttacaatgtcatcagcaaattccgcacaaagcaattattatgtatttgtattgtcggcgcattgttctccattctcggaacaaccgggattgtcgtgaattcagttatcggttgtatccccatcggcctcattgtggcacgatccttaaaatgggacgcagtcgcgggagccgctgttatatacatcggctgctacgctggatttaactccaccatattatcaccgtcaccgctcggtttatcacaatcaatcgcggagctccctcttttctcaggaatcggcctgcgagttgtgatatacatatgctttttgctgtcttctattatttatatctatttgtatacgagaaaattaaaaaaatcaaaagatgccagtgtgttaggaacagattggttccctgcggcaggaatgggcgaagccggtaaagaagaagatcagtcagtgccgtttaccgttcgccataagctgattttggctgtggcgggactctcacttgtcggatttttatacggcgctttgaagcttggctggtcagattcccaaatggctgcgacatttatttttatttctgtccttgccggtttaataggcgggcttgcggcgaacgatattgccaaaaccttcattacgggctgccaaagtcttgtatacggggcgctgattgtcgggatggcacgaagcatttccgttatccttgaaaatggaaagcttctcgatactgtcgtcaatgctttggcttcacttttggatggattcagcccgattgctggggcaatcggcatgtatatcgccagtgcgctgcttcattttctcatctcttcaggttctggcgaagccgttgtatttattccaatcctggcgccgctcgctgatttgatgggaatcacgagacaggttgcggttgaagcggttatgcttggagaaggggtcgtcaactgtgtgaacccgacatccggcgttctcatggcggtgcttgccgccagcggtattccgtatgtcaagtggctgcggtttatggtgccgcttgctctgatttggttcttgatcgggcttgtctttatcgtgatcggagtcatgatcaattgggggccgttttaacgattgctgcccgccggcttgtacggcgggcttttgagttattcattgcagaagcgcaggctgttattgtaacatgtaagccataagccattcgtaaaagtgcgggaggaaggtcatgaataatctgcgtaatagactttcaggcgtgaatgggaaaaataagagagtaaaagaaaaagaacaaaaaatctggtcggagaatgggatgatagcgggagcagttgctctgcctgatgtgatcatccgcggcattatgtttgaatttccgtttaaagaatggtctgcaagccttgtgtttttgttcatcattatcttatattactgcatcagggctgcggcatccggaatgctcatgccgagaatagacaccaaagaagaactgcaaaaacgggtgaagcagcagcgaatagaatcaattgcttgcgcctttgcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggtaatcaaataggctgtagctatttaatagctacagcctatttgcaactttctaagtttttctcaggatgattgatcacccgcggtgtaaaaaataggaataaaggggggttgacattattttactgatatgtataatataatttgtataagaaaatgagagggagaggaaacatgaagaaaccgttggggaaaattgtcgcaagcaccgcactactcatttctgttgcttttagttcatcgatcgcatcggctgctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcagtgccatggggaattagccgtgtgcaagccccagctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggccgggacgattgctgctttaaacaattcgattggcgttcttggcgtagcgccgagcgcggaactatacgctgttaaagtattaggggcgagcggttcaggttcggtcagctcgattgcccaaggattggaatgggcagggaacaatggcatgcacgttgctaatttgagtttaggaagcccttcgccaagtgccacacttgagcaagctgttaatagcgcgacttctagaggcgttcttgttgtagcggcatctgggaattcaggtgcaggctcaatcagctatccggcccgttatgcgaacgcaatggcagtcggagctactgaccaaaacaacaaccgcgccagcttttcacagtatggcgcagggcttgacattgtcgcaccaggtgtaaacgtgcagagcacatacccaggttcaacgtatgccagcttaaacggtacatcgatggctactcctcatgttgcaggtgcagcagcccttgttaaacaaaagaacccatcttggtccaatgtacaaatccgcaatcatctaaagaatacggcaacgagcttaggaagcacgaacttgtatggaagcggacttgtcaatgcagaagcggcaacacgctaaggtaataaaaaaacacctccaagctgagtgcgggtatcagcttggaggtgcgtttattttttcagccgtatgacaaggtcggcatcaggtgtgacaacgcgtgatctagaccagttccctgagcttccgtcagtcggatcccattgcggattttcctcctctaatatgctcaacttaaatgacctattcaataaatctattatgctgctaaatagtttataggacaaataagtatactctaatgacctataaaagatagaaaattaaaaaatcaagtgttcgcttctctctcacggagctgtaatataaaaaccttcttcagctaacggggcaggttagtgacattagaaaaccgactgtagaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatttcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataagtatatattttaaaaatatccacggttcttcaaatatttccccaagattttcctcctctaatatgctcaacttaatgacctattcaataaatctattatgctgctaaatagtttataggacaaataagtatactctaatgaccctataaaagatagaaggatccatagattaacgcgtggtacccggggatcctctaggccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaatttcggccgaatatcacaaatacatctgcagagttccaatacggagaaatcctgcaggatagtgcctccagagatgctgcatatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagacgacaggcgtttaaggatggagatcaattcacaatcggaaaaggagatccaatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattgagggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctgcaaggatgattaatgatgaacaaacatgtaaataaagtagctttaatcggagcgggttttgttggaagcagttatgcatttgcgttaattaaccaagggatcacagatgagcttgtggtcattgatgtaaataaagaaaaagcaatgggcgatgtgatggatttaccccacggaaaggcgtttgggctacaaccggtcaaaacatcttacggaacatatgaagactgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacaaatccggttgatatcctgacttacgcaacatggaaattcagcggcctgccaaaagagcgggtgattggaagcggcacaacacttgattctgcgagattccgtttcatgctgagcgaatactttggcgcagcgcctcaaaacgtacacgcgcatattatcggagagcacggcgacacagagcttcctgtttggagccacgcgaatgtcggcggtgtgccggtcagtgaactcgttgagaaaaacgatgcgtacaaacaagaggagctggaccaaattgtagatgatgtgaaaaacgcagcttaccatatcattgagaaaaaaggcgcgacttattatggggttgcgatgagtcttgctcgcattacaaaagccattcttcataatgaaaacagcatattaactgtcagcacatatttggacgggcaatacggtgcagatgacgtgtacatcggtgtgccggctgtcgtgaatcgcggagggatcgcaggtatcactgagctgaacttaaatgagaaagaaaaagaacagttccttcacagcgccggcgtccttaaaaacattttaaaacctcattttgcagaacaaaaagtcaactaaccgcaactttagagtaaagggctgattgtcaatgtgggagcagttgtatgatccgtttggaaacgagtatgtgagcgcacttgtggcgctcactccgattctcttttttcttttggctttaactgttttgaaaatgaaaggcattcttgcggcatttcttaccctagccgtcagtttcttcgtctccgtttgggcatttcatatgccggttgaaaaagcgatttcttctgttttgttaggaatcgggagcgggctgtggcccattggctacatcgtcctgatggcggtgtggctgtataaaatcgccgtgaaaaccgggaaatttaccattattcggtccagcattgccggcatttcgcctgaccaacgattacagctattattaattggtttttgttttaacgcgtttttagaaggcgcggccggttttggtgttccgattgcgattagtgcggcgctgctcgtcgaacttggttttaaaccgttaaaagcggcggcgctctgcttgattgcaaacgctgcctccggagcctttggggcgattgggattcctgtcatcacaggggcgcagattggtgatttgtctgctcttgagctgtctcggacattaatgtggacactgccgatgatctcatttttaataccattcctgcttgtattcttattagaccgaatgaaaggaatcaaacagacatggcccgctcttctggttgtgagcggtgggtatacagcggttcagacactgacaatggcggtgctcgggccggaattagcaaacattttggcggccttattcagcatgggcgggcttgccttcttcctccgcaaatggcagccgaaagagatttaccgcgaggaaggggccggcgatgctggtgagaaaaaggcataccgtgccgctgacattgcgagagcgtggtctcctttctacattttaactgcggcgatcaccatctgg